(19) United States
(12) Patent Application Publication
(10) Pub. No.: US 2010/0016268 A1
(43) Pub. Date: Jan. 21, 2010

(54) METHOD AND SYSTEM FOR DERMAL TISSUE TREATMENT

(75) Inventors: Robert Lyles, JR., Annapolis, MD (US); Robert Lyles, III, Towson, MD (US)

Correspondence Address: FOLEY & LARDNER LLP, 321 NORTH CLARK STREET, SUITE 2800, CHICAGO, IL, 60654-5313, US

(73) Assignee: Amergin, LLC

(21) Appl. No.: 12/554,810

(22) Filed: Sep. 4, 2009

Related U.S. Application Data
(63) Continuation-in-part of application No. 11/397,083, filed on Apr. 3, 2006, now abandoned.
Application No. 11/397,083 is a continuation-in-part of application No. 10/926,610, filed on Aug. 25, 2004, now Pat. No. 7,241,390.
(60) Provisional application No. 60/667,938, filed on Apr. 4, 2005.

(51) Int. Cl. A61K 31/56

(52) U.S. Cl. 514/180; 514/179

(57) ABSTRACT

A method and article of manufacture of treating abnormal biological tissue. The article includes use of electrochemically treated water in combination with a biologically active tissue treatment component, particularly for dermal tissue treatment. The electrochemical treatment method includes exposure of untreated water to halogen containing species, such as hypochlorous and hypobromous acid. The biologically active tissue treatment component can be a cortisone or clobetasol containing media.

### CROSS-REFERENCE TO RELATED PATENT APPLICATIONS

This application is a continuation-in-part of U.S. patent application Ser. No. 11/397,083, filed Apr. 3, 2006, which claims priority from continuation-in-part of U.S. patent application Ser. No. 10/926,610, filed Aug. 25, 2004, which claims priority from U.S. Provisional Application 60/667,938, filed Apr. 4, 2005, all of which are incorporated herein by reference in their entirety.

### BACKGROUND OF THE INVENTION

This invention relates to a method for controlling the quality of water. More particularly, the present invention relates to methods and apparatus for electrochemical treatment of water and for use of treated water for dermatological and other biological purposes.

In one aspect of the invention, the worldwide transfer and introduction of non-indigenous species by human activities is having significant and unwanted ecological, economic, and human-health impacts. For example, over the last decade, studies have found that ballast water has transported invasive marine species from their indigenous habitats, and far beyond the natural spread of the respective species. Organisms ranging from microscopic viruses to shellfish and aquatic plant life have been introduced into many U.S. waterways and ports. Each year more than 79 million metric tons of ballast water is released in the United States.

The past decade has seen extensive research of a handful of ballast water treatment options and similar levels of research have been devoted to charting the spread of AIS throughout the coastal and inland waterways of the United States. Disinfection of ballast water has not been achieved to date and current practices of ballast water management have been proven to be non-effective. The only existing mitigation option is mid-ocean ballast water exchange. Ballast water exchange is difficult to quantify in practice, cannot be safely performed on all transoceanic voyages, and by current definition cannot be conducted on voyages that take place within 200 miles of shore and in waters shallower than 2000 meters deep. Many experiments have been conducted on ballast treatment technology with respect to the general disinfection of water. Systems varying from ultra violet light radiation and mechanical filtration to thermal/heat exposure have been thoroughly investigated.

Despite all the research conducted to date, little has been accomplished with the disinfection of ballast water. Ballast water disinfection methodologies are limited by general trade industry guidelines that must be followed in order for a product to be feasible in the commercial shipping industry.

One example of such a treatment is described in U.S. Pat. No. 5,816,181 which discloses a thermal treatment method for the treatment of ballast water; U.S. Pat. No. 4,384,943 discloses a method and apparatus for the electrochemical treatment of fluid; U.S. Pat. No. 6,125,778 discloses an ozone treatment method of ballast water; U.S. Pat. No. 5,804,065 discloses an electric shock apparatus for the control of zebra mussels; U.S. Pat. No. 5,871,623 discloses an electrochemical disinfection apparatus for the treatment of water; U.S. Pat. No. 5,932,112 discloses a method for the treatment of ballast water by oxygenating and deoxygenating water; and U.S. Pat. No. 4,292,175 discloses an apparatus for the electrochemical treatment of ship's black water.

In another aspect of the invention, the dermal system of humans is subject to a variety of environmental and aging processes which cause degradation of the dermal system. In particular water is so integral to the human body and the surrounding environment that control of water in contact or embedded in the human body should be modified to improve the vitality of the dermal system. The use of treated water alone and in combination with selected dermal treatment media results in advantageous remediation and enhanced restoration of biological tissue, particularly dermal tissue.

### SUMMARY OF THE INVENTION

The invention was in part developed to meet the need for treatment of ballast water in the shipping industry as well treatment of water generally to decontaminate polluted water and create an improved water and an improved use for various biological purposes.

It is therefore one object of the invention to provide an improved water treatment option/improvement and method of use.

It is another object of the invention to provide an improved electrode design to treat conductive water.

It is a further object of the invention to provide a method for applying an improved electrical wave pattern for aqueous disinfection and/or treatment.

It is yet another object of the invention to apply the methods and apparatus of the invention to the treatment of any form of contaminated water to reserve the water to be environmentally safe.

It is a further object of the invention to provide an improved method, system and composition of matter involving water in contact with and ingested into the human body to improve the vitality of the human dermal system and other biological tissue.

The dermal system invention provides an improved alternate source of nano-nutrients to the skin directly by bypassing the gastrointestinal tract and the liver without traditional medications.

In chronic deficiency stages of skin disrepair there are strong effects of micro and macro nutrient deficiencies that create further susceptibility to morbidity of chronic disorder of the skin including increased susceptibility to damage from toxic environmental substances. Chronic disorders of the skin from excessive aging, secondary to toxic environmental substances, affect everyone. The magnitude of the health problems of the toxic disorder of the skin is a metabolic epidemic greater than obesity.

Approaches to providing skin health and cosmetic attractiveness depends upon age, gender, health status, diet, and environment exposure. Repletion of macronutrients through the use of the treated water invention can help the mesodermal layers of the skin maintain a healthy metabolism that effectively improves not only the cosmetic appearance of the skin, but also provides the much needed supplementation of nutrients to maintain health. All approaches to providing nano-nutrition to the skin encounters the problem of absorbability whether via the gastrointestinal tract with the first path degradation through the liver or other macromolecular entities that are placed on the skin and just “lay there.” The treated water product helps solve the problem of absorbability by creating an active stage product that uses both passive and active stages of the mechanism of diffusion by providing the metabolically live layers of the skin with much needed nutrition at the elemental nutrient stage.

It has long been observed that simple molecular substances, such as magnesium sulfate, separates into molecular and elemental phases, both of which use active and passive mechanisms of movement into and across the skin. The mechanism represents the most primitive form of nutrition for cellular structures. In another example, people worldwide have used copper bracelets in which small amounts of elemental copper can be actively transported through the cellular structure of the skin as a therapy for rheumatoid arthritis.

The water treatment invention yields a transformed state of elemental nutritional entities that are easily transported by passive and active mechanisms into the skin's cellular structure and are available for use as essential components of antioxidant enzymes that are effective in maintaining and improving the metabolic health of the skin, as well as, general nutrition.

Tests suggest that the end product has no discernable effect, either helpful or deleterious, to people whose skin is optimally healthy in the macro, micro, and nano-nutritional status. However, in skin which is deficient in elemental nano-nutrition or exhibits other unhealthy states, the effect is significant by which the skin obtains a healthy state. The effect in part is equivalent to immersing the skin in primal sea of nano-nutrition that provides a flow of nutrition and energy regulated by cellular mechanisms. The invention has been developed to utilize the primitive transdermal mode of nutritional source usage. In order for mechanisms to work best, three factors are typically present:

1. A state of metabolic deficiency in the skin
2. Source of energy—The use of treated water to alter the nutrients into an elemental ionic state which the skin can use
3. Nutritional source for the skin—The treated water method and use produces a nano-nutritional source for skin

These example objectives of the invention are non-limiting and these and other objects will become evident from the following Brief Description of the Drawings and from the Description of the Preferred Embodiments provided herein below:

### BRIEF DESCRIPTION OF THE DRAWINGS

FIG. 1 is a cross-sectional view of the electrode arrangement and position within the invention;

FIG. 2A is a view of the perforated plate electrode; FIG. 2B is a detailed view of this invention's electrode arrangement; and FIG. 2C is a detailed view of a non-effective electrode arrangement;

FIG. 3 is a drawing of the voltage waveform defined such that the current alternates and a high current limit is set so that the voltage is allowed to vary with changes in resistance; and

FIG. 4 illustrates residence time versus bacterial density for control and presence of hypochlorous acid.

### DETAILED DESCRIPTION OF THE PREFERRED EMBODIMENTS

In one form of the invention the formation of hypochlorous acid (HClO) by the present invention, applied to ballast water (and even all varieties of water for biological and non-biological purposes), is toxic to live water organisms. According to one embodiment of the invention, ballast water treatment can be performed and also be economically feasible, safe to the ship and crew, and environmentally friendly. In other forms of the invention, the methods and apparatus can be applied to various types of polluted and even “unpolluted” water having forms of unwanted contamination. The treatment enables creation of a highly desirable water-based fluid which has much improved properties, including use in dermal system treatment and other biological applications.

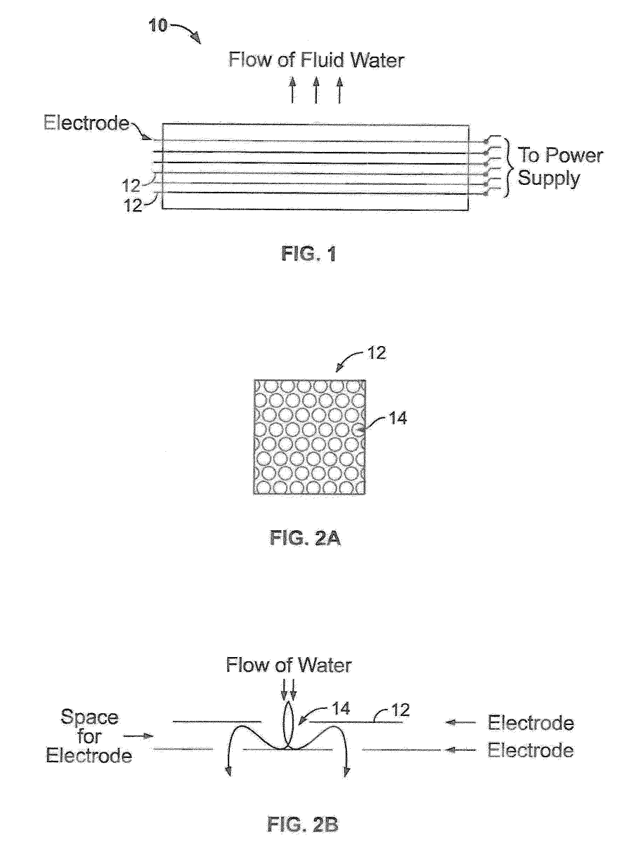

As shown in FIG. 1, the preferred form of the invention for water treatment and production of a useful end product includes an electrode array 10 such that the plurality of electrodes 12 are spaced in a series. The vertical position of the electrode array 10 directly exposes the passing organisms to the HClO formed during treatment. This positioning of the electrodes 12 ensures full water coverage over the electrodes 12 and the mixing of the anodic and cathodic chemicals formed during treatment. In addition, gravity is no longer a detracting feature because the vertical positioning creates a horizontal plane of hypochlorous acid during water treatment. Residence time of the water in the electrode array is also important to the formation of hypochlorous acid.

The preferred form of the invention provides the formation of HClO, and the following reactions occur in a saline solution at the anode of the electrodes 12:

2Cl⁻→Cl₂+2e⁻ (1)

2H₂O→O₂+4H⁺+4e⁻ (2)

H₂→2H⁺+2e⁻ (3)

Cl₂+H₂O→H⁺+Cl⁻+HClO (4)

In other forms of the invention, the reaction at the anode is not limited to Cl based constituents. For example, one can create hypobromous acid in the presence of bromine.

The anode has a positive charge; therefore, only chemical species with a negative or neutral charge will be local to the anode because like charges repel while opposite charges attract. As chemical species approach the anode, the electrostatic force between the negatively charged electrons of the chemical species and the anode increases. Chemical species local to the anode have a tendency of being oxidized. As a result, negatively charged species will become neutral and neutrally charged species will gain a net positive charge. The oxidation of water, for instance, requires the natural dissociation of one water molecule into one hydroxide ion (negative charge) and one hydrogen ion (positive charge). The hydroxide ion attacks a water molecule that subsequently yields hydrogen gas and a peroxide ion (negative charge). The peroxide ion spontaneously forms oxygen gas and a hydrogen anion. As the hydrogen anion is produced, it simultaneously binds to another hydrogen ion (produced during the initial dissociation of water), forming hydrogen gas. The hydrogen gas then undergoes reaction as shown in equation three recited above, which explains the four hydrogen ions in equation number two.

Ultimately, the most preferred lethal chemical species are Cl₂ and HClO (equation 4). These reactions occur during the anodic half cycle of the electrode; and during the cathodic half cycle, NaOH is formed. These two cycles alternate between electrodes, and this reversal of polarity helps provide a lethal chemical species in a zone that would ordinarily attract biofouling organisms.

As shown in FIG. 1, the electrodes are separated by nonconductive spacers. The distance from one electrode to the next is a function of the desired residence time and total electrical resistance. As the distance between the electrodes is increased, residence time for organisms increases; but the resistance in the electrical circuit also increases. This is problematic because the total power needed would increase beyond feasible use. Maintaining a long enough residence time for disinfection to occur can be achieved by stacking multiple pairs of electrodes. This arrangement creates parallel resistors, which reduces the overall resistance; and thus the overall power needed is also reduced (as is the residence time).

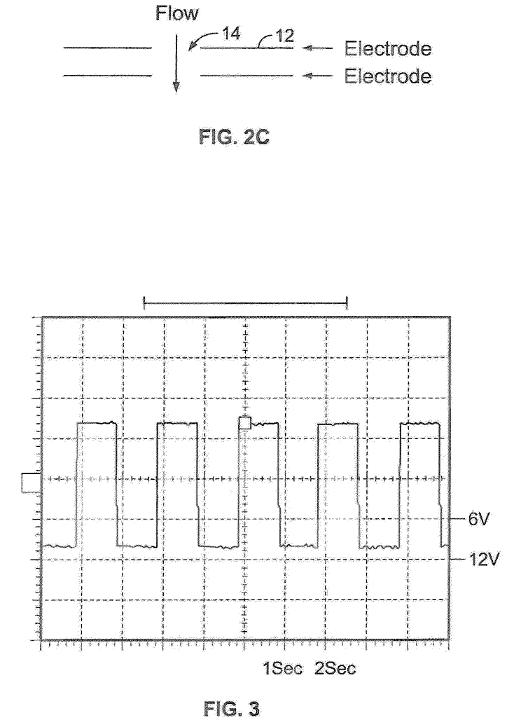

As shown in FIG. 2A for the most preferred embodiment, the electrode 12 has perforations 14 with approximately a 70% open area. The preferred pattern shown in FIG. 2A is advantageous to the electrode arrangement. The path of the water through the perforations 14 is important to the ability to complete disinfection across the electrode 12. As seen in the preferred embodiment of FIG. 2B, the water flow follows a serpentine path. In FIG. 2C, water flows directly through one perforation 14 to the next creating a tunnel effect, resulting in low disinfection. In the embodiment of FIG. 2B, the water flow is forced to come in contact with the electrode 12 for complete disinfection. The turbulent flow also ensures proper mixing of the water as it passes through the invention.

The invention can also include the attachment of a filtering device that may be useful to treat the aqueous solution (for example; for prevention of large suspended solids from being caught in the system and for chemical removal). Supplemental disinfection systems such as UV light treatment may also be included depending on the aqueous environments where the existing chemistry does not allow for desired disinfection rates.

In addition to electrode arrangement, a slow alternating current square wave applied to the electrodes 12 is able to prevent cathodic biofouling (see FIG. 3). A direct current (DC) can, in certain cases, result in problems with cathodic biofouling due to the accumulation of NaOH. The alternating current causes each electrode 12 to have a cathodic and anodic half cycle, which in turn interrupts the opportunity for fouling. This technique to increase the longevity of the electrodes 12 was first disclosed in U.S. Pat. No. 3,725,226, herein incorporated by reference.

The frequency of the slow alternating current (FIG. 3), which is used most preferably, ranges from 0.1 to 1 cycles per second. Cycles faster than 0.1 cycle per second generally do not allow enough time for the generation of a critical amount of HClO. Cycles slower than 1 per second generally cause irreversible cathodic biofouling. However, these less preferably ranges can still be useful in selected embodiments.

We have determined a current density range of 0.7 mA/cm2 to 20 mA/cm2 to be most effective at producing HClO in concentrations high enough to sterilize the water. Higher current densities increase the effectiveness when the flow rate is increased. However, increasing the velocity has shown a decrease in performance at the same current density. The negative side effect of increasing the current density beyond the stated range was the production of free Chlorine and Hydrogen gas.

The invention has numerous biological uses such as for aquarium treatment, water reuse treatment, healthcare instrument disinfection, municipal and commercial wastewater and blackwater treatment, industrial water treatment (for example, intake cooling pipes at nuclear power plants), and the above described marine commercial and pleasure craft blackwater treatment. In addition, as will be described in more detail hereinafter, the treated water, alone and in combination with conventional dermatological media, can provide enhanced restoration of abnormal biological tissue, such as dermal tissue.

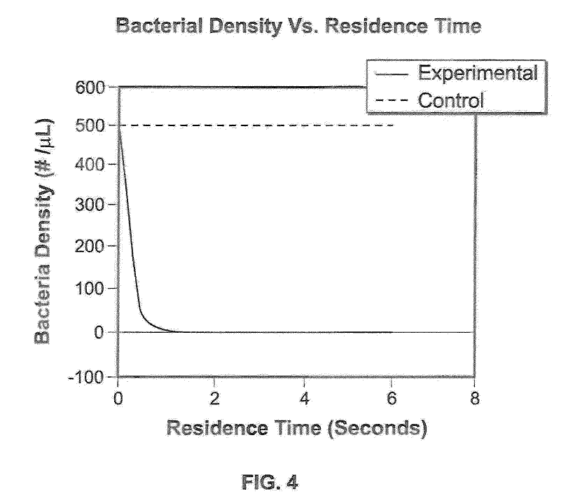

Example tests were performed using brackish water (obtained from the Chesapeake Bay, Md.). The tests measured bacteria densities of both the control and the treated water with the use of the invention. For all tests conducted; pH, conductivity, salinity, temperature, Chlorine concentration, and total dissolved solids of the water (pre and post treatment) were measured. There were no significant changes in overall water composition. The tests were run with residence times ranging from about 1 second to 6 seconds. Treated water had significantly fewer bacteria than the control (see FIG. 4).

### Example 1

| | | Conduc- | | | Chlorine | Total |
|---|---|---|---|---|---|---|
| | | tivity | Salinity | T | Conc. | Dissolved |
| | pH | (mS) | (ppt) | (° C.) | (ppm) | Solids (g/L) |
| Pre- | 7.55 | 24.7 | 15.0 | 22.0 | 0.0 | 14.44 |
| treatment | | | | | | |
| Post- | 7.59 | 24.7 | 15.0 | 22.0 | 0.4 | 14.45 |
| treatment | | | | | | |

This example provides one illustration of the present invention. Six electrodes are stacked to create three pairs of electrodes 12. The electrodes 12 are spaced ⅛ inch apart. This setup creates three parallel resistors as opposed to one resistor if only two of the electrodes 12 were to be used (water acts as the resistor). By having these parallel resistors, the resistance of the water is reduced to approximately one third of its original value according to Kirchoff's Law. This means that the electrical power required for only one pair of the electrodes 12 would be three times greater than in this current model according to Ohm's Law. A slow A.C. waveform is employed with a frequency of 1 Hz and a current limit of 0.75 amps. By setting the current limit such that the voltage would not peak at the defined voltage, the voltage may vary with slight resistance changes in the water. In this example, the voltage would be about 6 volts.

The following Examples 2-4 illustrate the restorative effect of the treated water on abnormal biological tissue and also the substantial advantages of combining the treated water with various conventional dermal tissue treatment media, such as for dermal tissue using cortisone- and clobetasol-based formulations and/or other curative or anti-inflammatory chemical species.

The Chesapeake Bay water is pumped from an untreated holding tank through the electrode array 10 into a clean tank. The flow rate of the water through the system is 8 liters per minute. Water samples are taken at various points along the transfer as well as throughout the duration of the transfer. Samples are cultured for evidence of living organisms and the result was a marked reduction in contaminating species.

### Example 2

### Forehead Skin with Winter Eczema

A. Seawater was first treated in accordance with the method described herein. Usage was for a 7 day session or until endpoint achieved, applied Invention treated seawater solution to affected area twice daily for 7 days.

B. Cortisone 1% (OTC) Cream, usage for 7 day session or until endpoint achieved, applied Cortisone 1% Cream to affected area for 7 days.

C. Treated seawater+Cortisone 1% Cream, usage for 7 day session or until endpoint achieved, applied first Invention treated seawater to the affected area and air dried, this was followed by an application of Cortisone 1% Cream to the same affected area.

Skin before treatment is described as red, dry, scaly, patches of itchy erythematous facial skin with flaky defoliation at mechanical contact and epidermal thickening.

1. Skin after treatment A for 5 days presented with resolution of pretreatment symptoms of eczema. The skin in the eczema areas returned to a hydrated, soft, state without scaling defoliation, itching, or epidermal thickening. However, at 5 days there remained a slight degree of redness without irritation. At completion of the 7 day therapy, the redness of the new skin development was resolving

2. Skin after treatment B for 7 days presented with partial resolution of the most severe symptoms, namely a reduction in scaling, itching, with the skin appearing more hydrated. However, evidence of defoliation continued but softened by the cream base of the treatment.

3. Skin after treatment C for 7 days presented with good to excellent resolution of symptoms after 3 days; at the 5 day interval of therapy, there was excellent to complete resolution of the presence of the eczematous skin areas; after 7 days of therapy the shin was presented with a well hydrated, normal healthy appearance with no noticeable differences between the eczematous areas and the normal areas of the forehead skin.

### Example 3

### Plaque Psoriasis on Bilateral Knees after Traumatic Injury

A. Treated seawater was used for a 7 day session or until endpoint achieved, applied the treated seawater solution to the affected area twice daily for 7 days.

B. Clobetasol (prescription) cream, usage for 7 day session or until endpoint achieved, applied clobetasol 0.05% Cream to affected area for 7 days.

C. Treated seawater+clobetasol 0.05% cream, and was used for a 7 day session or until endpoint achieved, applied treated seawater to the affected area and air dried, this was followed by an application of Cortisone 1% Cream to the same affected area.

Skin before treatment is described as red/white, dry, silvery scaled, thickened patches of itchy erythematous skin over the anterior knee inferior to the patella (bilaterally) with flaky defoliation at mechanical contact.

1. Skin after treatment A for 7 days presented with partial resolution of pretreatment symptoms of psoriasis. The skin in the psoriatic areas slowly progressed with a reduction in scaly hypertrophy thickening to a hydrated, softer state without itching and reduced epidermal thickening. However, at 5 days there remained a slight degree of redness without irritation. At completion of the 7 day therapy, the redness of the plaqued areas was resolving with skin progressing to more normal pathology.

2. Skin after treatment B for 7 days presented with partial resolution of the most severe symptoms, namely a reduction in scaling, itching, with the skin appearing more hydrated. However, evidence of defoliation continued but softened by the cream base of the treatment. Treatment was continued for 14 days before what appeared to be a progression to remission (short term with rapid return after stopping treatment)

3. Skin after treatment C for 7 days presented with good to excellent resolution of symptoms after 5 days; at the 7 day interval of therapy, there was substantial excellent resolution of the presence of the psoriatic skin areas; after 14 days of therapy the skin was presented with a well hydrated, healing, healthy appearance with little noticeable differences between the psoriatic diseased areas and the normal areas of the skin except for the expected scar-state discoloration.

### Example 4

### Erythematotelangiectatic Rosacea—Subtype I

Treated seawater was used for 7 to 30 day sessions or until endpoint achieved-abatement of signs and symptoms, applied treated seawater solution to affected area twice daily for the duration of each session.

Skin before treatment is described as having the traditional symptoms of episodic flushing and redness of the cheeks usually occurring daily in the early evening. Traditional treatments, creams, etc., had not been effective.

The patient initially uses treated seawater for study periods of 7 days to 2 weeks and notice some changes at the 2 week milestone such as reduced frequency defined as not experiencing everyday flushing and redness, an example was possibly experiencing episodes of rosacea symptoms only 6 out of 7 days per week. When period of treatments were extended up to a full month of daily treatments, the frequency of daily episodes of flushing and redness decreased with the extended period of treatment.

The treatment periods that extended for greater than 1 month resulted in the absence of episodes of rosacea symptoms as long as the Invention treated seawater therapy was continued on a daily basis.

While preferred embodiments have been illustrated and described, it should be understood that changes and modifications can be made therein in accordance with one of ordinary skills in the art without departing from the invention in its broader aspects.

### Example 5

An isotonic solution (0.9% saline) created from natural seawater and distilled water was treated with the method described herein. The solution containing the activated ionic species was applied to the skin and a self-regulated transport of the solution occurred through the skin without the requirement of external driving forces, electrolytic, or otherwise. The transport across the skin was evident in the smooth muscle relaxation and de-vascularization of the skin, rendering normal skin tone.

### Example 6

A hypertonic solution (3% saline) of natural seawater obtained from the Atlantic Ocean was treated with the method described herein. The solution containing the activated ionic species was applied to the skin and a self-regulated transport of the solution occurred through the skin without the requirement of external driving forces, electrolytic, or otherwise. The transport across the skin was evident in the smooth muscle relaxation and de-vascularization of the skin, rendering normal skin tone.

### Example 7

A hypertonic solution (30% saline) of natural seawater combined with dehydrated sea salts was created and treated with the method described herein. The solution containing the activated ionic species was applied to the skin and the solution passed through the skin via the self-transport mechanism which required no external driving forces, electrolytic, or otherwise. The transport across the skin was evident in the smooth muscle relaxation and de-vascularization of the skin, rendering normal skin tone.

What is claimed is:

1. An article for treatment of biological tissue, consisting essentially of an electrochemically pretreated water in combination with a biologically active tissue treatment component not treated electrochemically, thereby providing an article for application to dermal tissue and having curative chemical species.

2. The article as defined in claim 1 wherein the tissue treatment component comprises a dermal treatment component.

3. The article as defined in claim 1 wherein the electrochemically pretreated water includes a residual halogen containing chemical species.

4. The article as defined in claim 3 wherein the halogen containing species is selected from the group consisting of chlorine and bromine.

5. The article as defined in claim 1 wherein the electrochemically pretreated water has been treated by a method comprising exposure of starting water to a constituent selected from the group of hypochlorous acid and hypobromous acid.

6. The article as defined in claim 1 wherein the tissue treatment component comprises an anti-inflammatory constituent.

7. The article as defined in claim 6 wherein the anti-inflammatory constituent is selected from the group consisting of a cortisone containing material and a clobetasol containing material.

8. The material as defined in claim 7 wherein the cortisone containing material comprises about 0.1-2% cortisone disposed in a biocompatible media.

9. The material as defined in claim 7 wherein the clobetasol containing material comprises about 0.01-0.1% clobetasol in a biocompatible media.

10. The material as defined in claim 1 wherein the tissue treatment component comprises an internal tissue treatment component.

11. The material as defined in claim 10 wherein the internal treatment component comprises a digestive tissue treatment component.
